(19) United States
(12) Patent Application Publication
(10) Pub. No.: US 2017/0009207 A1
(43) Pub. Date: Jan. 12, 2017

(54) IN-VITRO BIOREACTOR

(71) Applicant: New York University, New York, NY (US)

(72) Inventors: D. Barrett Shamir, Beachwood, OH (US); Jared Schwartzentruber, Astoria, Queens, NY (US)

(73) Assignee: New York University, New York, NY (US)

(21) Appl. No.: 15/210,636

(22) Filed: Jul. 14, 2016

Related U.S. Application Data
(63) Continuation-in-part of application No. PCT/US2015/011215, filed on Jan. 13, 2015.
(60) Provisional application No. 61/927,356, filed on Jan. 14, 2014.

(51) Int. Cl. C12N 5/077; C12M 1/36; C12N 13/00; C12M 1/00; C12M 1/34; C12M 1/42; C12M 1/12

(52) U.S. Cl. CPC C12N 5/0657; C12M 35/04; C12M 35/02; C12M 41/48; C12M 25/02; C12M 35/08; C12M 29/10; C12M 41/46; C12N 13/00; C12N 2529/00; C12N 2527/00; C12N 2501/10

(57) ABSTRACT

An in vitro chamber for mimicking the mechanical and electrical forces found in vivo under physiological or pathological state. A chamber provides conditioning stimuli. The conditioning stimuli replicate a pathology, including the ability to replicate a diseased pathology. The chamber is configured to allow the additional stimuli to test physical, chemical or electrical stimuli impact on cells experiencing the particular pathology. Pharmaceuticals may be tested ex-vivo on cells exhibiting a pathology in an environment mimicking the in-vitro environment. Physical components such as pacemakers may also be tested on such cells in the chamber.

### CROSS-REFERENCE TO RELATED APPLICATIONS

The present application is a continuation-in-part of International Patent Application No. PCT/US2015/011215, filed Jan. 13, 2015, which claims priority to U.S. Patent Application No. 61/927,356, filed Jan. 14, 2014, all of which are incorporated herein by reference in their entirety.

### FIELD OF THE INVENTION

The present invention generally relates to in-vitro models.

### BACKGROUND OF THE INVENTION

To date there are many static in-vitro models of the heart that use cardiomyocytes, transfected cells or differentiated stem cells to mimic physiological aspects of the heart. They achieve this modeling through inducible means; pharmacologically, chemically, and other biochemical and molecular manipulations. To date there is no cell culture models that provide mechanical forces (e.g. pressure and volume) and electrical stimulation in a synchronous fashion like found in the heart. These stimuli put the cells through the same physiological conditions as if they were in-vivo.

### SUMMARY OF THE INVENTION

One embodiment of the invention relates to an in vitro cardiac chamber for mimicking the mechanical and electrical forces found in the heart.

Another embodiment relates to an apparatus for simulating a heart. The apparatus includes cell chamber for receiving cells. The apparatus further includes a control chamber comprising a control system. The cell chamber and the control chamber are removably connectable. A cell membrane is engageable with the housing. A chamber layer is disposed within the housing and engagable with the cell layer, the chamber layer including a pressure and volume control system.

Another embodiment relates to a method of simulating a cardiac environment for cell growth comprising applying a conditioning stimulus to the cell layer in the cell chamber, the conditioning stimulus selected from the group consisting of chemical, physical, electrical and combinations thereof and extracting the cell layer from the cell chamber.

Another embodiment, a nontransitory computer-readable memory having instructions thereon, the instructions comprising instructions for selecting a pathology to mimic; applying a plurality of conditioning stimuli to the cell layer in the cell chamber, the conditioning stimulus mimicking the selected pathology and selected from the group consisting of chemical, physical, electrical and combinations thereof; monitoring the plurality of conditioning stimuli; and monitoring the cell layer.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A illustrates a detached view of an embodiment of an in vitro cardiac chamber; FIG. 1B illustrates an exploded section view of an embodiment of a bioreactor; FIG. 1C illustrates an exploded view of an embodiment of a bioreactor.

FIG. 2 illustrates an attached view of an embodiment of an in vitro cardiac chamber with all possible volume and pressure configurations upon the cell layer.

FIGS. 3A-3C illustrate cross-sectional views of a bioreactor; FIG. 3A illustrates where the media pressure is equal to the air pressure; FIG. 3B illustrates where the air pressure is greater than the media pressure; FIG. 3C illustrates where the air pressure is less than the media pressure.

FIG. 4A illustrates on embodiment of a system with tubing, media reservoir, and media restrictor valve to demonstrate the flow of media and variables that will effect changes in pressure and hemodynamic's of the system; FIG. 4B illustrates an alternative embodiment of a system with the bioreactor, media reservoir, perfusion pump, and restrictor valves.

FIG. 5 illustrates characteristics of a natural in vivo heart in comparison to a prior art device and an embodiment of an in vitro cardiac chamber of the present invention.

FIG. 6 illustrates a flow chart depicting operation of one embodiment.

FIG. 7 illustrates a computer system for use with certain implementations.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

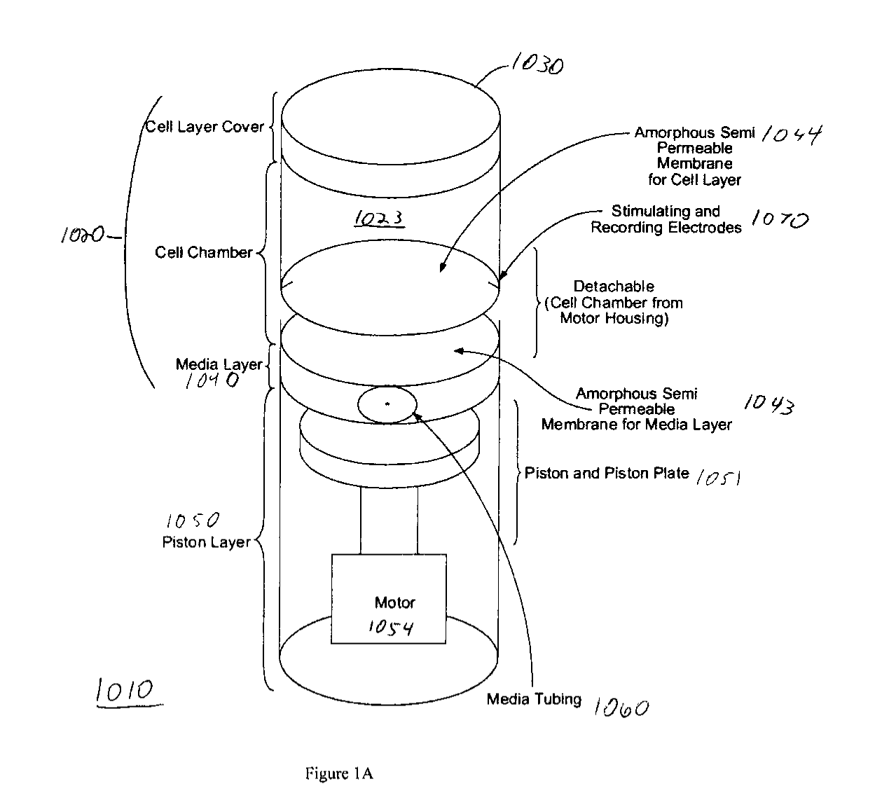

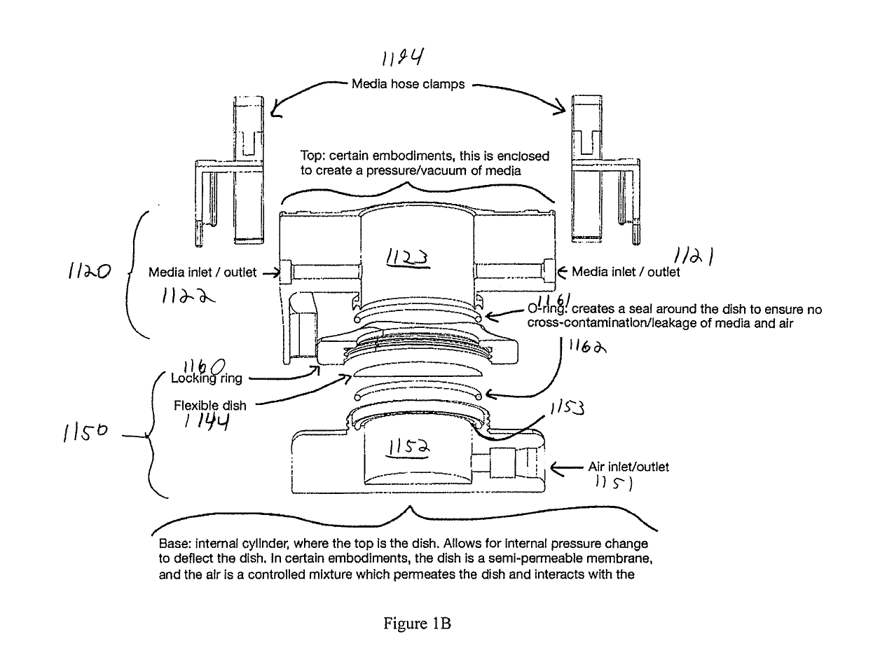

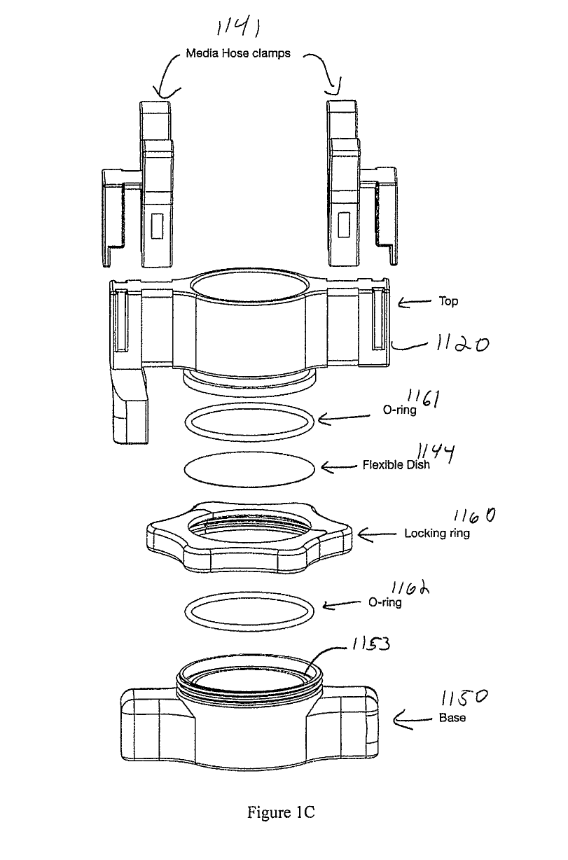

One implementation relates to an in-vitro model that simulates the environment; physical and electrical forces that a cell would experience in vivo, as physiological or pathological. A bioreactor is provided for housing the cell(s). FIG. 1A illustrates an embodiment of a bioreactor 1010. The embodiment includes a media chamber 1021. The media chamber 1021 may be part of a upper media portion 1020. The upper media portion 1020 includes a cell layer cover 1030. In one embodiment, the upper media portion 1020 includes as a lower boundary a cell layer 1040. In another embodiment, the cell layer 1040 is separate from the upper media portion 1020. The cell layer 1040 is disposed over an lower control portion 1050. Alternatively, the cell layer 1040 may be integral with either the lower control portion 1050 or the upper media portion 1020. The cell layer 1040 may comprise a flexible disk or membrane 1044 on or in which the cells are disposed. Alternatively, the disk 1044 may be separate from the cell layer 1040, where the cell layer 1040 provides a media layer and the disk 1044 provides the support for the cells (see FIG. 1A). An actuator 1050 is, in one embodiment shown in FIG. 1A, provided in the lower control 1050 and includes a piston 1051 and motor 1054. The lower control portion 1050 also includes a lower control portion chamber 1052. Media may be supplied, such as circulated, to the bioreactor 1010 by media tubing 1060. The lower control portion chamber 1052 and the media chamber 1021 may be filed, to controlled pressures, by media. In one embodiment, the upper media portion 1020 is removably attachable to lower control portion 1050, such as to the actuator 1051.

In one embodiment illustrated in FIG. 1B, a bioreactor 1110 includes an upper media portion 1120 and a lower control portion 1150 with a cell layer 1140 disposed therebetween. The upper media portion 1120 includes a media inlet 1121 and a media outlet 1122 with an upper media chamber 1123, such as disposed there between. The lower control portion 1150 includes an inlet 1151 and a lower control chamber 1152. The inlet 1151 may be an inlet/outlet or there may be a separate outlet (not shown). In the embodiment of FIGS. 1B and 1C, the media (or cell) layer 1140 comprise a flexible disk 1144 disposed at an upper opening of the lower control portion 1150. The cells are disposed on or in the cell layer 1140. A support ridge 1153 may be provided in the lower control portion 1150 to support the flexible disk 1144 about a periphery. The flexible disk 1144 is able to flex upwards, i.e. into the upper media chamber 1123, or downwards, i.e. into the lower control chamber 1152.

In one embodiment, shown in FIGS. 1B and 1C, the upper media portion 1120 is removably engagable with the lower control portion 1150, such as, for example, by a threaded mechanism, a tongue-and-groove mechanism, or other suitable mechanical connections. A locking ring 1160 may be used to threadable secure the upper media portion 1120 to the lower control portion 1150. Preferably, seals, such as o-rings 1161, 1162, are provided at interfaces between the components. Such o-rings may be seated within recessed grooves, such as a groove on the support ridge 1153.

The embodiment of FIG. 1A utilizes a mechanical system 1080 and an electrical system 1070. The embodiment of FIGS. 1B-C utilizes a perfusion system 1190, a mechanical stretch apparatus 1180, and an electrical system 1070. The in vitro model includes one or more of a perfusion system 1190, used here generally to describe delivery of materials to the cell layer 1140, such materials can include blood, cell growth media, air, water, all generally referred to herein as “media,” a mechanical system 1080, which applies mechanical forces, such as stretch, strain, volume change, and pressure, and an electrical system 1070, which applies current to the cells.

In one embodiment, the in vitro model is of the heart. The in vitro model may change pressure and/or volume independently to induce in-vivo like characteristics of cardiomyocytes, such as through a combined impact of the perfusion system 1190 and the mechanical system 1080. The in vitro model may change the rate and level of electrical stimulation by the electrical system 1070 to induce in-vivo like characteristics of cardiomyocytes. The stimulation within the in vitro environment and forces induces cells to behave as if they are in vivo. For example, such simulation may induce in-vivo cardiomyocyte morphology, biochemistry, cell signaling, electrical conduction properties, membrane potentials, transcription levels, and cytokinetics.

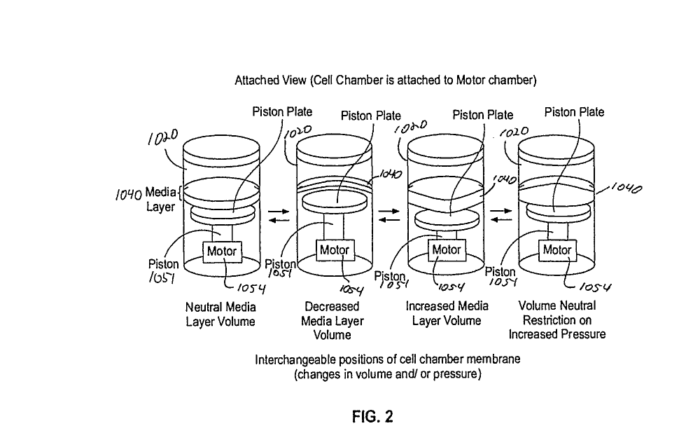

In one embodiment, the in vitro model is a cardiac chamber device. This cardiac chamber device would contain an area to simulate the heart, such as to seed and plate cardiomyocytes/cells. For implementations utilizing cells, the simulated heart area may be referred to as the cell layer (a cell layer or the semipermeable membrane seeded with). The cell layer 1040 is illustrated in FIG. 1A as detached from the remainder of the cardiac chamber device. The cell layer is illustrated in FIG. 2 as attached to, and integral with, the remainder of the cardiac chamber device. FIG. 2 also shows possible configurations and physical stressors that can be implemented due to changes with volume and/or pressure to the cell layer. In the embodiment of FIGS. 1B-C, the cell layer 1140 is supported by a flexible disk 1144. The cell layer 1140 may be a membrane or matrix in which the cells are disposed or a surface on which the cells are disposed. The cell layer 1140 may be impermeable or may be permeable to one or more of liquids or gases. When the cell layer 1140 deflects downward it increases the volume within the upper chamber, thus allowing more media to enter (or more media at the same pressure where the media may otherwise be compressible). In addition, the cell layer 1140 which is composed of some semi-permeable membrane could allow for different concentration of air flow to pass depending on if it is static, deflected in the positive direction or in the negative direction.

Adjacent this simulated heart area is a system for simulating the action of any of the chambers of the heart (atrium and ventricles) under physiological or pathological conditions. In one implementation, cells are seeded onto a membrane 1044. In one particular implementation, the simulated heart chamber comprises an amorphous, semi-permeable membrane 1044 underneath the simulated heart area. The membrane may comprise glass or plastic. For example, in one implementation, the membrane comprises a polypropylene, such as a flexible polypropylene. In one exemplary implementation, depending on the requirements of the cells utilized, a protein matrix is provided on the membrane 1044. For example, the selected protein matrix may be of a known type beneficial for a selected type of cell, such as to promote one or more of adhesion of cells to the membrane 1044, growth or cell signaling. The semi-permeable membrane serves as the material upon which cells are seeded. It also serves a sight of diffusion from which nutrients from the media chamber 1021 can enter the cell layer 1040 and vice versa. The cell layer 1040 would be fluid filled with a nutrient containing medium to promote cell survival. In one embodiment, bioreactor 1010 is a simulated heart chamber layer is configured to inflate and deflate, thus changing volume and pressure, mimicking the expansion and contraction of any of the chambers of the heart. In another embodiment, the bioreactor 1110 undergoes deflection by action of the mechanical system 1080, but the mechanical system 1080 does not appreciably contribute to fluid flow. Rather, fluid flow is primarily driven by the perfusion system 1190.

In one embodiment, the membrane 1044 may be configured to allow for ease of extraction of cells from the membrane 1044. The cells may be extracted as a tissue, i.e. as a connected group of cells. Several different strategies can be used, alone or in combination, to facilitate the extraction of cells. In order to graft new cardiac muscle tissue into damaged hearts it would be advantages for the cells to be extracted as a single unit. The membrane 1044 (or cell layer 1040), in one embodiment, is pre-coated with a protein matrix that helps to keep the cells together as a single piece of tissue. In another embodiment, another option is using a porous (semi permeable) biodegradable plastic (like that found in heart stents) as the membrane 1044 to be put below the cells. Extract the cells with this plastic and then implant the cells.

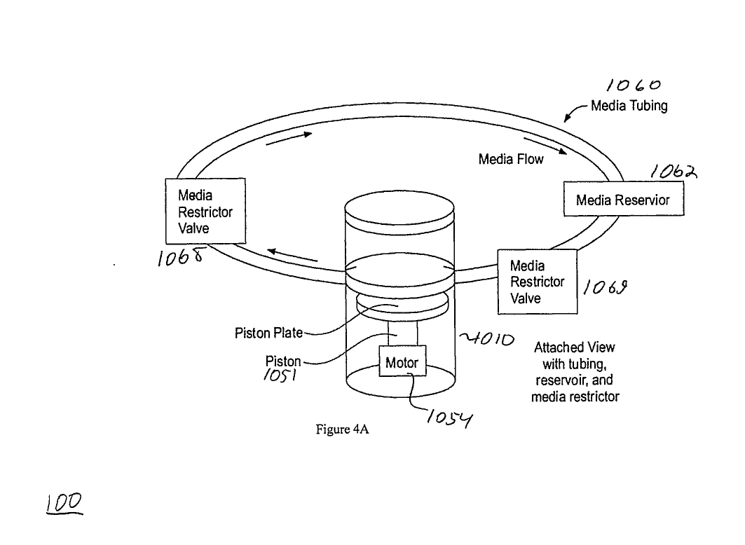

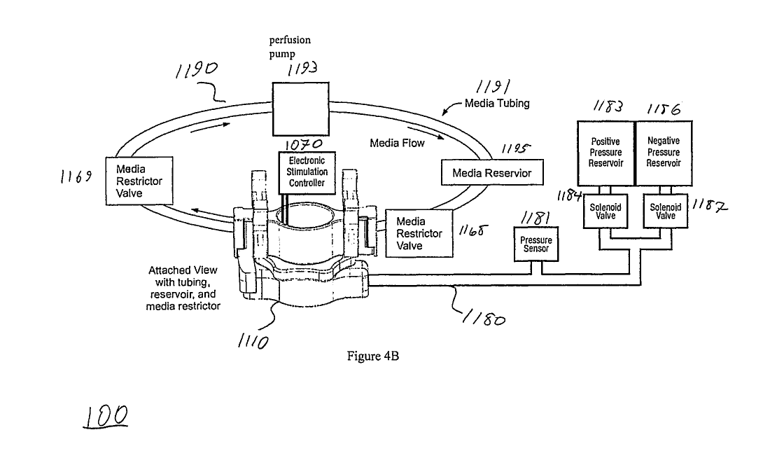

In one embodiment, the mechanical system 1080 comprises a mechanical stretch apparatus 1180. For example, FIG. 4B illustrates an embodiment where a mechanical stretch apparatus 1180 is connected to the inlet 1151. The mechanical stretch apparatus 1180 includes a pressure sensor 1181 as well as a positive pressure reservoir 1183 and an associated positive (or high) pressure valve 1184 and a negative pressure reservoir 1186 and associated negative (or low) pressure valve 1187. The pressure within the lower control chamber 1152 is controlled. The inlet/outlet 1151 of the control portion 1150 is connected to the high-pressure 1183 and the low-pressure reservoir 1184. In certain embodiments, a separate motor is used to create high pressure and low pressure within each reservoir 1183, 1186. In another embodiment, the pressure differential is created by one or more pumps, including two-way pump (not show). The outlet of each reservoir is fed through a valve, e.g. a solenoid valve. The outlet of each valve is combined and connected to a hose. The hose outlet is connected to the bioreactor 1010/1110. The valves open and close in a controlled manner, switching the pressure within the hose and bioreactor between high and low. In certain embodiments, the pressure is maintained in the reservoirs by a pressure sensor in each reservoir. The motor duty cycle is adjusted relative to the current pressures and predefined pressures. In some embodiments, a pressure sensor is placed along the hose between the bioreactor and the valves. A predefined pressure cycle is compared to the pressure read from the pressure sensor, and the motor duty cycle is adjusted accordingly.

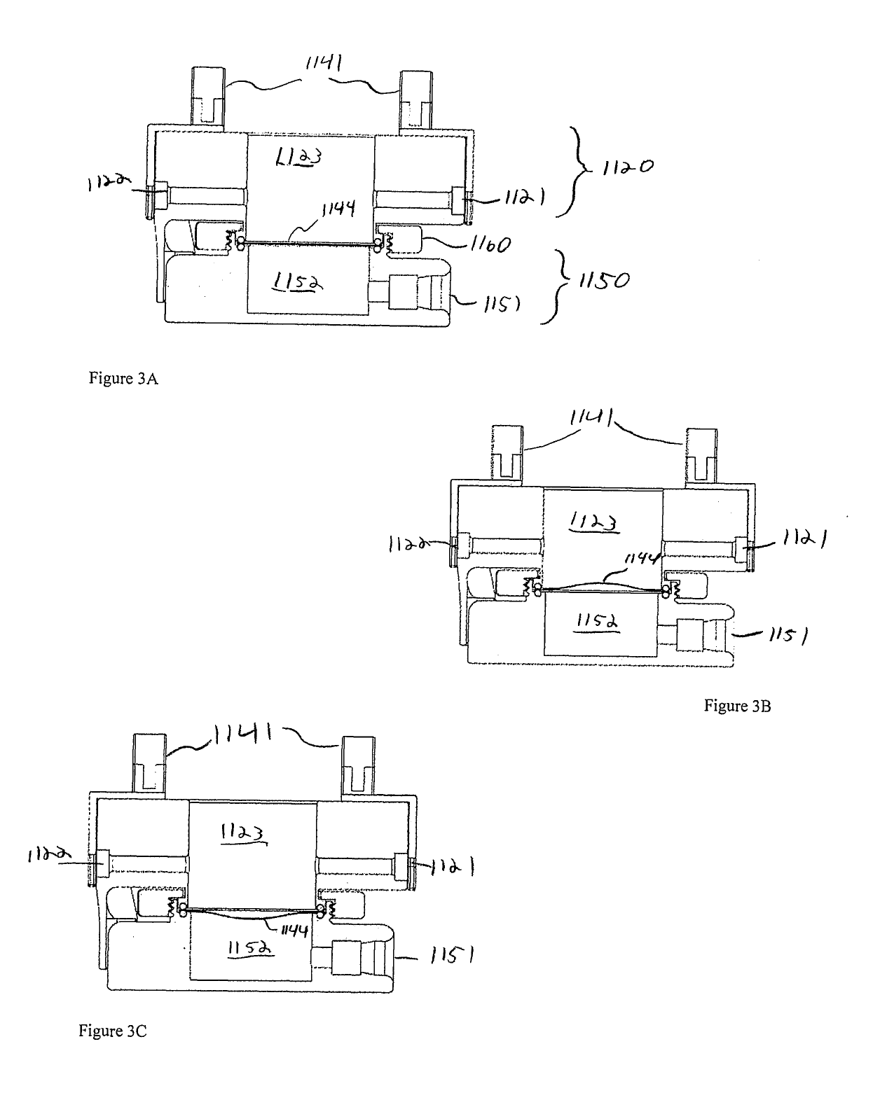

In one embodiment, the mechanical stretch mechanism utilizes pressure differentials between the lower control portion 1150 and the upper media portion 1120. FIGS. 3A-3C illustrate action of the mechanical stretch apparatus 1180 and the resultant action of the disk 1144. When pressure between the upper media chamber 1123 and the lower control chamber 1152 are equal, the disk 1144 is “at rest” or unflexed. When the pressure in the lower control chamber is greater (FIG. 3B) the disk is stretched and flexes into the media chamber 1123 away from the higher pressure. When the pressure in the lower control chamber is less (FIG. 3C) the disk is stretched and flexes into the control chamber 1152 away from the higher pressure. The mechanical stretch apparatus changes the volume within the bioreactor, which may change the total amount of volume the cell is exposed to.

In the embodiments of FIGS. 1B-C, 3A-C, and 4B, the bioreactor 1110 is in communication with a perfusion system 1190. The perfusion system 1190 comprises a media line 1191, connected at one end to the outlet 1122 and at another end to the inlet 1121. A perfusion pump 1193 is in fluid communication with the media line 1191 and, thus, the upper media portion 1120. One or more media house clamps 1194 may be utilized. A media reservoir 1195 may be in communication with the media line 1191 to provide a reservoir of media for the perfusion system 1190. The perfusion system 1190 may be “open” or “closed”. Additional materials, such as air (including specific gases such oxygen or carbon dioxide) may be directly infused into the circulating media of a closed perfusion system. For an open system, the circulating media may be exposed at a specific interface to allow exchange and interaction with the atmosphere (which itself may be controlled, such as part of an incubator or the like).

The perfusion system 1190 controls the rate of media added into the bioreactor 1110. While the mechanical stretch apparatus 1180 may slightly expand (or decrease) the volume, the perfusion system 1190 has the dominate impact on the pressure in the bioreactor and the cardiac chamber through controlling the flow of media. As an example, as the membrane 1044 or disk 1144deflects downward (by the mechanical stretch apparatus) the bioreactor 1010/1110 is filled up with media (as activated by the perfusion system 1190).

The systems allow for individual control of volume, pressure, and electrical conduction upon the cardiac cells. This is caused by a combination of factors. First the mechanical stretch apparatus 1180 deflects the disk 1144 downward, increasing the volume of the chamber. Second, the perfusion system 1190 will provide increased media into the chamber 1123. This will occur to the point where the pressure then increases within the chamber 1123, as measured by pressure sensors. Finally when a threshold pressure is met, the mechanical stretch apparatus 1180 will deflect the disk 1144 upwards. The perfusion system 1190 will be allowed to move the media to its target destination towards the outlet of the bioreactor.

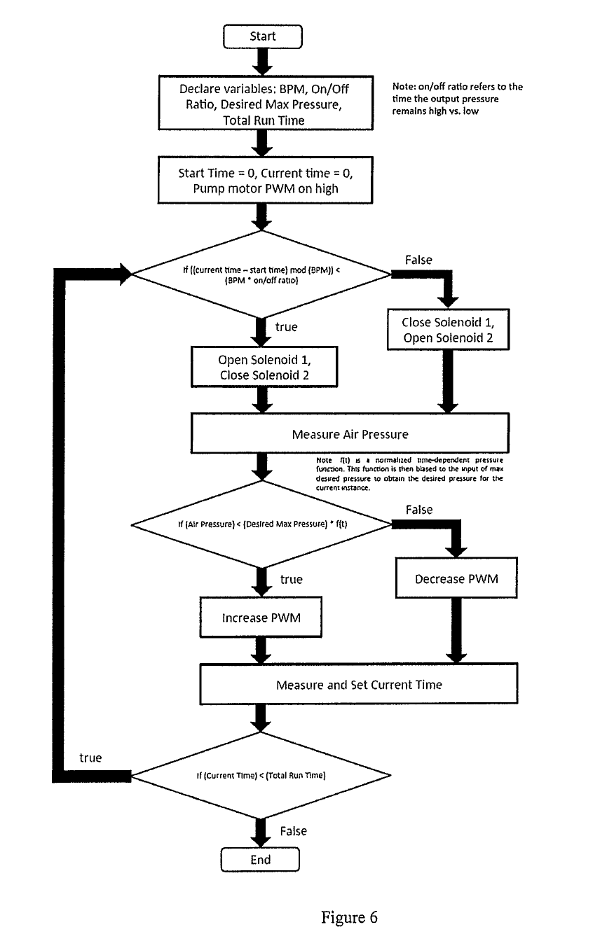

FIG. 6 illustrates one method of operating a system as shown in FIG. 4B. In one embodiment, the process, starting off the synchronous function of the system, may be initiated by electrical stimulation from the electrical system 1070. Once the initiation occurs, the pump 1193 is turned on in the perfusion system 1190, beginning a fill up phase. The restrictor valve 1168 at the media inlet 1121 will be open, while the restrictor valve 1169 at the media outlet 1122 is closed. As the chamber 1123 is expanding, volume increases, stretch (of the cell layer 1140) increases. This allows media into the chamber 1123. The incoming restrictor valve 1168 is open at this time. The outgoing restrictor valve 1169 is closed. The perfusion system 1190 will be activated in the positive direction into the device. The mechanical stretch apparatus 1180 initiates so the cell layer 1140 will deflect downwards (into the control chamber away from the media chamber) due to a lower pressure. Ultimately enough media will fill up the entire apparatus, causing a slight increase in pressure. The mechanical stretch apparatus 1180 and the perfusion system 1190 may perform sequentially (with either being first) or simultaneously.

When a pressure threshold is met, the mechanical stretch apparatus 1180 reverses the pressure differential, resulting in the cell layer 1140 deflecting upwards (into the media chamber), reducing the volume and increasing the pressure more, to a new maximum pressure.

In one embodiment, the occurrence of this maximum pressure signals the end of the inflow phase and the start of the outflow phase. The relative opening of the restrictor valves is reversed to allow the perfusion system 1190 to remove media from the upper media chamber 1123. When that max pressure is hit, the outgoing restrictor valve 1169 will open and the incoming valve 1168 will close, decreasing the pressure in the chamber 1123, and allow for the outflow of media to the media reservoir 1062 and perfusion system 1190. The media chamber 1123 is emptied or evacuated to a desired volume, effectively resetting the system for the start of a new inflow phase the process will start again with the electrical stimulation of the cells,

In one embodiment, the inlet 1021 and outlet 1022 of the media chamber 1052 are controlled by one or more valves 1068, 1069. These valves 1068, 1069 (or valves 1168, 1169 of the embodiment of FIG. 4B) maybe controlled open/close, rather than a check valve. The direct control of the opening or closing of the cardiac chamber valves allows for further control of the overall environment within the cardiac chamber, whereas a one-way check valve merely opens at the set pressure threshold. Further, the valves can be adjusted or replaced with different size or types of valves to simulate various pathological conditions.

In another embodiment, rather than a perfusion system 1190 and a mechanical stretch apparatus 1180, the pressure (volume) and fluid flow are controlled only by the mechanical system 1080, which comprises a piston 1051 is movable by a motor 1054 and engageable with the media chamber 1021. The motor 1054 also may allow for an actual contraction of the cardiomyocytes as it can provide direct mechanical force upon the cell layer 1040, like the heart which expands and contracts in an active manner (see FIG. 2). Fluid loads may vary with volume between chambers 1052, 1021 (FIG. 1A) or chambers 1123, 1152 (FIGS. 1B-C). This fluid load variation exerts force and the cells are not exposed to just the sheer stress due to pressure alone. In addition to the sheer stress, the chamber volume will vary, thus exposing the cells to both a change and pressure and in the volume of the associated chamber.

In one embodiment, a pressure gauge or control, such as one or more restrictor valves, is included. For example, such a pressure control may act as an in vitro equivalent for a mitral and/or semi-lunar valve. Further, in one embodiment pressure control may be independently associated with each chamber (see FIGS. 4a, 4B).

Structurally, the pressure control valve may be part of the tubing exiting the chamber 1021 and leading medial to the loop. The pressure control valve could be turned on and off manually, and replaced with a variety of valves of different diameter allowing different amount of media load to enter the cell layer 1040. Preferably, these valves are unidirectional like mitral, tricuspid, and semi lunar valves. These different sized valves could better mimic certain pathological states as well.

In one embodiment, all of the necessary media is stored within the media portion 1020/1120 and the cell layer 1040/1140 and any tubing or fluid lines. In such an embodiment, a media reservoir 1062, such as media bottles or external media sources are not included. In one embodiment, the media bottles or external media sources are connected to the main housing of the device. The described components provide a device that gives the seeded cells an in vitro environment that mimics an in vivo environment. A media reservoir 1062 may be placed in communication with the media tubing 1060, such as shown in FIG. 4A. For example, the device gives the seeded cells an apparatus that mimics heart expansion and contraction in synchrony with electrical stimulation. In one implementation, the device accommodates spontaneous activation of one or more cells without stimulation, such as the spontaneous firing of a cardiomyocyte.

In one embodiment, the electrical system 1070 provides electrical stimulation to the cell layer 1040 and the cells therein/thereon. In one embodiment, two simple electrodes are positioned in the media, such as in the media chamber. The electrodes apply galvanic stimulation within the media of the device. In another embodiment, the electrodes are in direct contact with the cell layer 1140, including in direct contact with the cells in one particular implementation. Yet another embodiment, the electrical system 1070 includes a wiring system either embedded or slightly above the cells which will mimic the purkinje fiber system of the heart, so electrical conduction only occurs within a certain pattern upon the heart cells. In this embodiment, the electrical stimulation could be applied via galvanic stimulation, direct contact, or embedded wiring within the cell layer, the disk or membrane, or the cells themselves.

This electrical stimulation system 1070 applies pulses of electrical stimulation that will operate at similar frequencies, voltages, and currents of the heart in physiological or pathological states. This will be conducted in synchrony with the other perturbations and follows the cardiac cycle.

In one embodiment a transducer is provided as part of the control system to convert one form of energy into a mechanical movement of the cell layer 1040. A piezoelectric component may be used. Further, the cell layer 1040 may be moved to mimic a heart chamber by increasing and decreasing the fluid and or air in the cell layer 1040, in the media chamber 1021, and/or in the control chamber 1052.

Certain embodiments of an in vitro cardiac device allow a quick and easy way to monitor changes in heart cells ability to contract and to generate action potentials. In one embodiment, overall conduction of the entire cell layer 1040 is monitored. Information regarding resistance, currents, and voltage can also be provided. One method of monitoring is to utilize patch clamping, such as patch clamping of cardiomyocytes. In one implementation, the membrane potentials/action potentials are indirectly monitored, such as by measuring the overall systems changes in membrane potentials. Similar to an ECG, in this implementation, a first electrode is below the cell layer in the media and a second electrode above in the chamber layer, at the opposite ends. Thus treating both electrodes as leads, like when measuring ECG, the potential can be monitored. In one implementation, monitoring would be done through the use of a computer system that will be hooked up to the device and transmit directly to a computer where all the data can be actively monitored.

In one implementation, the device is configured to allow for adjustment to the beats per minute as simulated. For example, a variable control with regard to the stroke of the piston may be provided or with regard to the inflow and outflow cycle. The stroke speed and/or length may be adjusted.

The in vitro cardiac chamber device would allow for relatively easy manipulation, such as by, but not limited to, cardiac tissue pharmacologically, electro-physiologically, gas exchange, and mechanical perturbations. The in vitro cardiac device may be used for pharmaceutical research to test new target drugs in an easy and quick way without the need of generating labor-some transgenic animal models. Further, in one implementation the in vitro cardiac device can be utilized as a fast, high throughput, screening mechanism by utilizing the device an in-vitro system that mimics in-vivo like conditions. This in vitro cardiac device can also mimic any of the chambers of the heart as each chamber of the heart contains different ejection loads (fluid output of chamber, which is a function of volume and pressure variables), mechanical perturbations, and electrical stimulations. Further, the in vitro cardiac device may be used to study cellular effects of physiological and pathological changes in cardiomyocytes including but not limited to; hypertension, tachycardia, bradycardia, ischemia, arrhythmias.

In one implementation, the device includes an additive intake. The additive intake is configured to allow for control of pharmacological changes such as can be inducted by adding certain drugs/reagents into the cell media. In a further implementation, gas exchange would be achievable based on the use of gas permeable tubing and/or the use of a gas exchange associated with the upper chamber of the device.

In a further implementation, the device may be placed within an incubator or configured to function in cooperation with an incubator (not shown).

In one implementation, the in vitro heart chamber device is able to simulate both the diastolic and systolic activity of an in vivo heart. The in vitro heart chamber device would induce the morphological changes upon the cardiomyocytes using mechanical loads and electrical stimulation in synchrony which would be similar to the order and magnitude of the events of the actual heart. During the cardiac cycle there are two phases: diastole and systole. During diastole the given heart chamber (in this case the ventricle) fills up with blood, gradually increasing the total volume of the chamber and with little change in pressure. It is at this point where the cardiomyocytes experience increased levels of shear stress. It is only when the heart is in systole (contraction) where volume dramatically decreases and at the same time the pressure increases. The mechanical perturbations of systole would be preceded by electrical stimulation in a similar sequence of events as in the heart, where the electrical stimulation would cause depolarization of the cardiomyocytes. A cardiomyocyte may depolarize and ultimately start an action potential when its membrane potential increases over a certain threshold (which varies depending on what part of the heart). At this point, sodium channels and subsequently calcium channels open up allowing these ions to flow into the cell. Ultimately there is a refractory period when these channels can stay open no longer and must begin to close. Typically, this is when an action potential hits its peak membrane potential. Finally, potassium channels begin to open, repolarizing (decreasing the membrane potential) back to the steady state. The electrodes in the device will act as the SA or AV nodes. A group of cells in the heart that act as “pacemakers,” and being the starting point in a cascade of heart muscle contraction. These action potentials act as a wave as it moves down the heart. In one implementation, a strip is utilized for the stimulation of the membrane. In another implementation, two electrodes are utilized.

This pressure increase would then allow the blood to enter either the aorta or pulmonary artery by bypassing any of the major valves (e.g. mitral valve or semi-lunar valves). In this in vitro heart model the pressure increase would be great enough to pass through the valve places on the inlet and oulet from the media chamber (i.e. through the restrictor valves). This valve would be mimicking the role of the major valves of the heart. This valve would be unidirectional and restricting fluid flow until the appropriate pressure has been met (which would be achieved during systole).

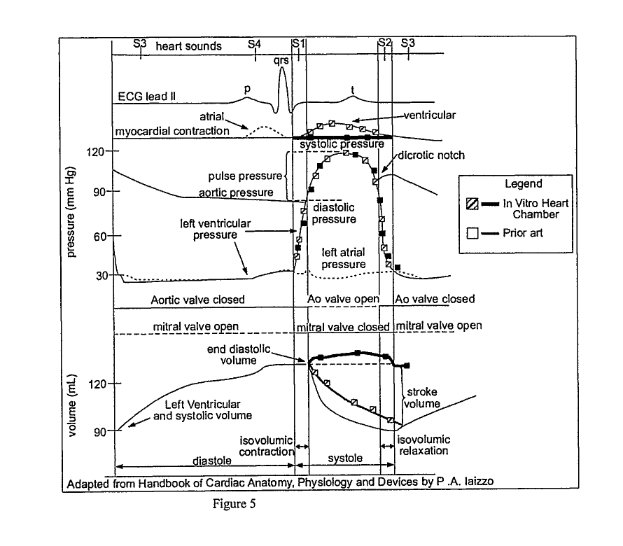

In the in vitro heart chamber device both volume and pressure could be independently controlled to mimic similar physiological mechanical changes in pressure and volume during the cardiac cycle. That is, each of volume and pressure can be independently controlled. Both the volume over time and pressure over time of the in vitro heart chamber device closely mimic that of a natural heart. Specifically, the profile pressure over time and volume over time for the in vitro heart chamber device indicate a diastole phase and a systole phase. FIG. 5 illustrates pressure and volume for an in vivo heart, a prior art device, and a device in accordance with an embodiment of the present invention.

In one embodiment, the in vitro heart chamber device includes an electrical component to induce the morphological changes upon the cells. For example, the cell layer may be provided electrical stimulation prior to mechanical stimulation. As can be seen in FIG. 5, the QRS wave (ventricular contraction) directly precedes the contraction (and changes in volume and pressure). The prior art does not allow for electrical stimulation with control of mechanical perturbations (e.g. volume and pressure). The presence and control of all three is important as the heart requires electrical and mechanical stimulation to induce proper morphological changes and function. In one embodiment, the in vitro heart device synchronously induces both the mechanical and electrical stimulation to the cell layer, such as where electrical stimulation would precede mechanical contraction.

In a further embodiment, the in vitro heart chamber device provides chemical areas of stimulation, for example two such areas. Most cells are polar and have a basal and apical ends or at minimum receive different chemical stimulants from different areas along the same cell. The in vitro heart chamber device may provide apical stimulation from cells from the paracardium (via conditioned medium). In one implementation, the paracardial stimulation is provided components in the cell layer. For example, the media directly on top of the cells and the media flowing underneath cells would be that of similar nutrients as the coronary arteries/capillaries. Thus, the in vitro heart chamber device may provide chemical stimulation from two different locations and source.

In another implementation, the forces that are simulated in the model may reflect forces beyond those experienced in vivo. For example, the conditions applied in the model may exceed the typical conditions experienced in vivo or may even exceed the known limits of conditions experienced in vivo. As such, the model may be used to “stress test” cells in conditions more extreme than would be experienced in vivo. In one implementation, the device may be utilized to test known pathological states (which may not be “normal” in vivo states), for example tachycardia. To mimic tachycardia, the device utilizes an increased rate of a “beat”, i.e. cycling the piston faster by increasing the speed of the associated motor. In an alternative implementation, the device may operate out of “normal” in vivo state with regard to electrical conditions, such as changing the rate or amount of stimulation of the electrodes to simulate SA or AV node malfunction in certain arrhythmias. Further, in another implementation relating to abnormal states, the device may utilize a change in volume and pressure to also elicit conditions similar to in vivo conditions associated with certain diseases, such as, Atrial Enlargement. Also changing the size of the media restricting valves to several diameters (larger or smaller) could elicit or help exemplify other diseases such as, valvular stenosis, valvular insufficiency, certain congenital valve diseases, etc. Further, changing the CO₂/O₂ levels of the system can help elicit an ischematic event. In addition, changing a combination of variables could help elicit the cells in the system to a wide variety of diseases.

In another implementation, the in vitro model may simulate a diseased state, such as a diseased heart's function. The system may be operated in a physiological or a pathological mode. That is, the system can recapitulate and condition cells under most pathological conditions or to a particular patients exact physiological conditions.

In one embodiment, the cells conditioned in the chamber can be extracted for biochemical assays.

In one embodiment, the in vivo heart chamber device uses mechanical stimulation, including pressure and volume change, to induce in-vivo characteristics of cardiac function and is an assay that is fast and reproducible compared to in-vivo models.

In one embodiment, the cell layer 1040/1140 is provided as a removable component, such as a cartridge (not shown). The remaining portions of the device may be reused, such that a cell layer cartridge can be replaced without the need for an entirely new device. The cell layer cartridge may include a growth medium, an electrode, and an amorphous semi permeable membrane. The cell layer cartridge's amorphous semi permeable membrane is adjacent and engageable with the chamber layer's amorphous semi permeable member. The tubing may be gas permeable in one embodiment. The device may be housed in an incubator with temperature and gas exchange controlled.

In one embodiment, the in vitro heart chamber device can be used to create cardiac tissue for implantation to an organism. Although cardiac cells can be grown in laboratory using known techniques, such cardiac cells are grown in an environment very different from the actual in vivo environment that such cells function. In one method, the heart chamber device is utilized to condition cardiac tissue exposing the tissue to chemical, physical, and electrical stimuli and environmental conditions that replicate those of an organism, for example the cells in the heart chamber device are exposed to a physiologically identical set of conditions. The conditioning may constitute one or more of chemically (trophic and other growth factors), physically (volume and pressure manipulation), and electrically (electrical stimulation) conditioning.

The replicated conditions can be selected to be those of a typical organism such as a human. The replicated conditions applied to condition the cells may be physiological or pathological, allowing for varied study or healthy or diseased states or conditioning for use in a patient with a healthy or diseased state. In one embodiment, compounds such as drugs or additives may be added to the device. For example, users of this device add the compounds into the media supplied on the cell layer or into the media reservoir. Further, the media and cells can be extracted at various time points to determine biochemistry, cytokinetics, and expression patterns,

Further, in one embodiment, pace maker or other similar heart control devices can be used in combination with cardiac tissue in the device. For example, the device can be used to test and mount new methods, algorithms, and devices for pace makers as well. The physiological impact of devices such as pacemakers alone or in combination with electrical stimulation and pharmacological manipulations can be studied.

Ideally, the heart chamber device is configured to replicate the conditions of a specific organism, such as the intended human transplant patient, so the cardiac tissue grown in vitro is grown in an environment identical or substantially identical to the ultimate in vivo environment where the tissue will be implanted. Typically one of the biggest barriers of transplant is the body rejecting the new tissue. In this case, the cells conditioned by the devices described herein can circumvent this in certain embodiments. Further, the new tissue grown in the device can be manipulated through molecular or pharmacological means to make the patient's body think the new tissue is “self” tissue, reducing or eliminating the risk of rejection. On a functional level being able to differentiate and condition cells will give them a better shot at functioning more similarly to the patient's body with less chance of arrhythmias or Atrial fibrillation caused by misfiring or cardiomyocytes due to lack of synchrony of cardiac tissue action potential firing. For example if the new tissue is eliciting action potentials at a higher frequency then the patient's heart this could cause dysynchronous activity or even an unwarranted contraction of the heart.

In one embodiment, conditioned cells can be transferred to a recipient patient. In one method, the conditioned cells are disassociated using a cell detachment solution, such as one including proteases like trypsin or accutase. The conditioned cells may be disassociated for removal from the heart chamber device as a single tissue or as cells for suspension in a solution. As a result of the conditioning within the device, for example the different stresses placed on the cells, there will be biochemical changes as the actin and myosin filaments levels would fluctuate due to the stress put on the cells. Further, there will be functional changes that will be different as a result of biochemical, and molecular changes within the cardiac tissue. Second connexin ion channels as well as other ion channel production would change and this would impact the ability of these cells to fire action potentials and contract as a result. A static model without the mechanical perturbations enabled by the device described above, but only pharmacological manipulation would not meet the same changes in all these protein levels and subsequent functional output (action potentials). A membrane, such as described above, may be used to transfer cells from the in vitro heart chamber to a recipient patient. It is believe that will push less defined tissues to react in the manner that is physiologically specific and relevant for the patient. Less defined tissue will not contain the molecular and proteins necessary to keep up with the rest of the heart. This pre conditioning by the described device will increase rate of recovery as the cells integrate faster as they need less time to become conditioned in the heart as well.

In one embodiment, the in vitro heart chamber device is in communication with a computer system to control the in vitro heart chamber device. The computer system may monitor the characteristics of the in vitro heart chamber device, such as electrical stimulation, pressure, and volume. The computer system may also allow for adjustment and control of one or more individual characteristics of the in vitro heart chamber device. In one embodiment, the system provides monitored information regarding cells in the in vitro heart chamber device. In response to the monitored information, individual conditions within the in vitro heart chamber device can be adjusted to alter the cells. For example, the ratio of carbon dioxide to oxygen as well as the absolute levels of both can be controlled within the device. On the device itself the volume and pressure could be manipulated, as well at the rate at which the heart chamber contracts and expands.

The computer may include programmed instructions to alter one or more conditions of the in vitro heart chamber device when the monitored information includes certain information or a particular combination of information. It should be appreciated that the device is scalable and that for certain automated embodiments all that would be necessary is the initial seeding of the cells and the occasional media reservoir change. Otherwise the system can be automated by the computer system to electrically stimulate, physical perturbations,

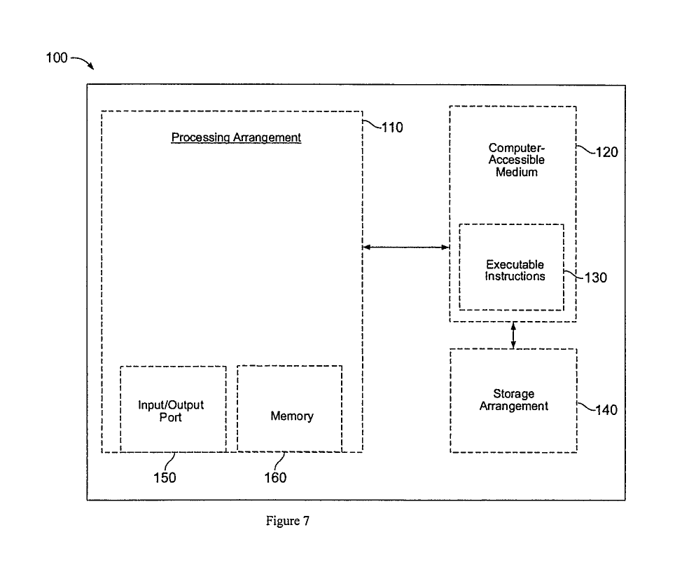

As shown in FIG. 7, e.g., a computer-accessible medium 120 (e.g., as described herein, a storage device such as a hard disk, floppy disk, memory stick, CD-ROM, RAM, ROM, etc., or a collection thereof) can be provided (e.g., in communication with the processing arrangement 110). The computer-accessible medium 120 may be a non-transitory computer-accessible medium. The computer-accessible medium 120 can contain executable instructions 130 thereon. In addition or alternatively, a storage arrangement 140 can be provided separately from the computer-accessible medium 120, which can provide the instructions to the processing arrangement 110 so as to configure the processing arrangement to execute certain exemplary procedures, processes and methods, as described herein, for example.

System 100 may also include a display or output device, an input device such as a key-board, mouse, touch screen or other input device, and may be connected to additional systems via a logical network. Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An apparatus for simulating a heart comprising:
a bioreactor having a media portion with a media chamber therein, and a lower control portion; the bioreactor having a cell layer for receiving cells; and
a mechanical system and an electrical system in communication with the media layer, the mechanical system configured to exert physical stress upon the cells and the electrical system configured to exert electrical stimulation to the cells.

2. The apparatus of claim 1 wherein the electrical system includes electrodes conductively connected to the cell layer and configured for electrical stimulation of the cells.

3. The apparatus of claim 1 wherein the mechanical system includes a piston and a motor and wherein the cell layer comprises a membrane.

4. The apparatus of claim 3 wherein the membrane is positioned above the piston and adjacent the cell chamber when connected and movement of the piston causes fluid to flow through the cell membrane and cause sheet stress at the cells.

5. The apparatus of claim 1, further comprising a perfusion system in fluid communication with the bioreactor.

6. The apparatus of claim 5, wherein the lower control portion includes a control chamber and the mechanical system comprises a mechanical stretch apparatus and the cell layer includes a flexible dish.

7. The apparatus of claim 6, wherein the media portion comprises a media inlet and a media outlet each connected to the perfusion system.

8. The apparatus of claim 7, wherein the mechanical stretch apparatus comprises a positive pressure reservoir and a negative pressure reservoir in communication with the control chamber.

9. The apparatus of claim 1, wherein the electrical system further comprises a pacemaker in communication with the cell layer for electrical stimulation of the cells.

10. The apparatus of claim 1, wherein the cell layer further comprises a semi-permeable cell layer membrane configured for receiving the cells and for receiving media.

11. A method for providing conditioned cardiac environment cells growth comprising:
applying a conditioning stimulus to the cell layer in the cell chamber, the conditioning stimulus selected from the group consisting of chemical, physical, electrical and combinations thereof; and
extracting the cell layer from the cell chamber.

12. The method of claim 11, wherein the conditioning stimulus is physical and comprises mechanical stimulation.

13. The method of claim 12, wherein the mechanical stimulation includes a change in a cell layer adjacent the cell layer.

14. The method of claim 12; wherein the conditioning stimulus is electrical stimulation.

15. The method of claim 14 wherein conditioning comprises synchronized application of the physical stimulation and application of the electrical stimulation.

16. The method of claim 14, wherein conditioning comprises application of electrical stimulation before application of the mechanical stimulation.

17. The method of claim 11 wherein the chemical stimulation includes a growth factor.

18. A nontransitory computer-readable memory having instructions thereon, the instructions comprising:
selecting a physiology or a pathology to mimic;
applying a plurality of conditioning stimuli to the cell layer in the cell chamber, the conditioning stimulus mimicking the selected pathology and selected from the group consisting of chemical, physical, electrical and combinations thereof;
monitoring the plurality of conditioning stimuli; and
monitoring the cell layer.

19. The computer-readable memory of claim 18, wherein the pathology is selected and wherein the pathology is a diseased state.

20. The computer-readable memory of claim 18 further comprising instructions for controlling a pacemaker in communication with the cell layer.
